(19) United States
(12) Patent Application Publication
(10) Pub. No.: US 2012/0265074 A1
(43) Pub. Date: Oct. 18, 2012

(54) PROVIDING THREE-DIMENSIONAL ULTRASOUND IMAGE BASED ON THREE-DIMENSIONAL COLOR REFERENCE TABLE IN ULTRASOUND SYSTEM

(75) Inventors: Kyung Gun NA, Seoul (KR); Sung Yun Kim, Seoul (KR)

(73) Assignee: SAMSUNG MEDISON CO., LTD.

(21) Appl. No.: 13/445,505

(22) Filed: Apr. 12, 2012

(30) Foreign Application Priority Data
Apr. 12, 2011 (KR) 10-2011-0033913

(51) Int. Cl. A61B 8/00; G06K 9/00

(52) U.S. Cl. 600/443; 382/131

(57) ABSTRACT

There are provided embodiments for a three-dimensional ultrasound Image based on a three-dimensional color reference table. In one embodiment, an ultrasound system comprises: a storage unit for storing a three-dimensional color reference table for providing colors corresponding to at least one of intensity accumulation values and shading values throughout depth; and a processing unit configured to form volume data based on ultrasound data corresponding to a target object and perform ray-casting on the volume data to calculate intensity accumulation values and shading values throughout the depth, the processing unit being further configured to apply colors corresponding to the at least one of the calculated intensity accumulation values and the calculated shading values based on the three-dimensional color reference table.

### CROSS-REFERENCE TO RELATED APPLICATIONS

The present application claims priority from Korean Patent Application No. 10-2011-0033913 filed on Apr. 12, 2011, the entire subject matter of which is incorporated herein by reference.

### TECHNICAL FIELD

The present disclosure generally relates to ultrasound systems, and more particularly to providing a three-dimensional ultrasound image based on a three-dimensional color reference table in an ultrasound system.

### BACKGROUND

An ultrasound system has become an important and popular diagnostic tool since it has a wide range of applications. Specifically, due to its non-invasive and non-destructive nature, the ultrasound system has been extensively used in the medical profession. Modern high-performance ultrasound systems and techniques are commonly used to produce two-dimensional or three-dimensional ultrasound images of internal features of target objects (e.g., human organs).

The ultrasound system may provide a three-dimensional ultrasound image including clinical information, such as spatial information and anatomical figures of the target object, which cannot be provided by a two-dimensional ultrasound image. The ultrasound system may transmit ultrasound signals to a living body including the target object and receive ultrasound echo signals reflected from the living body. The ultrasound system may further form volume data based on the ultrasound echo signals. The ultrasound system may further perform volume rendering upon the volume data to thereby form the three-dimensional ultrasound image.

When performing volume rendering upon the volume data based on ray-casting, it is required to calculate a gradient corresponding to each of the voxels of the volume data. Since a substantial amount of calculations and time are required to calculate the gradient corresponding to each of the voxels, the gradient is calculated at a preprocessing stage prior to performing volume rendering. However, a problem with this is that volume rendering (i.e., ray-casting) cannot be performed in a live mode for rendering the volume data acquired in real-time, based on the gradient.

### SUMMARY

There are provided embodiments for providing a three-dimensional ultrasound image based on a three-dimensional color reference table for providing colors corresponding to at least one of intensity accumulation values and shading values throughout depth.

In one embodiment, by way of non-limiting example, an ultrasound system comprises: a storage unit for storing a three-dimensional color reference table for providing colors corresponding to at least one of intensity accumulation values and shading values throughout depth; and a processing unit configured to form volume data based on ultrasound data corresponding to a target object and perform ray-casting on the volume data to calculate intensity accumulation values and shading values throughout the depth, the processing unit being further configured to apply colors corresponding to the at least one of the calculated intensity accumulation values and the calculated shading values based on the three-dimensional color reference table.

In another embodiment, there is provided a method of providing a three-dimensional ultrasound image, comprising: a) forming volume data based on ultrasound data corresponding to a target object; b) performing ray-casting on the volume data to calculate intensity accumulation values and shading values throughout the depth; and c) applying colors corresponding to the at least one of the calculated intensity accumulation values and the calculated shading values based on a three-dimensional color reference table for providing colors corresponding to at least one of intensity accumulation values and shading values throughout depth.

The Summary is provided to introduce a selection of concepts in a simplified form that are further described below in the Detailed Description. This Summary is not intended to identify key or essential features of the claimed subject matter, nor is it intended to be used in determining the scope of the claimed subject matter.

### BRIEF DESCRIPTION OF THE DRAWINGS

FIG. 1 is a block diagram showing an illustrative embodiment of an ultrasound system.

FIG. 2 is a block diagram showing an illustrative embodiment of an ultrasound data acquisition unit.

FIG. 3 is a schematic diagram showing an example of acquiring ultrasound data corresponding to a plurality of frames.

FIG. 4 is a flow chart showing a process of forming a three-dimensional color reference table.

FIG. 5 is a schematic diagram showing an example of volume data.

FIG. 6 is a schematic diagram showing an example of a window.

FIG. 7 is a schematic diagram showing an example of polygons and surface normals.

FIG. 8 is a flow chart showing a process of forming a three-dimensional ultrasound image.

### DETAILED DESCRIPTION

A detailed description is provided with reference to the accompanying drawings. One of ordinary skill in the art should recognize that the following description is illustrative only and is not in any way limiting. Other embodiments of the present invention may readily suggest themselves to such skilled persons having the benefit of this disclosure.

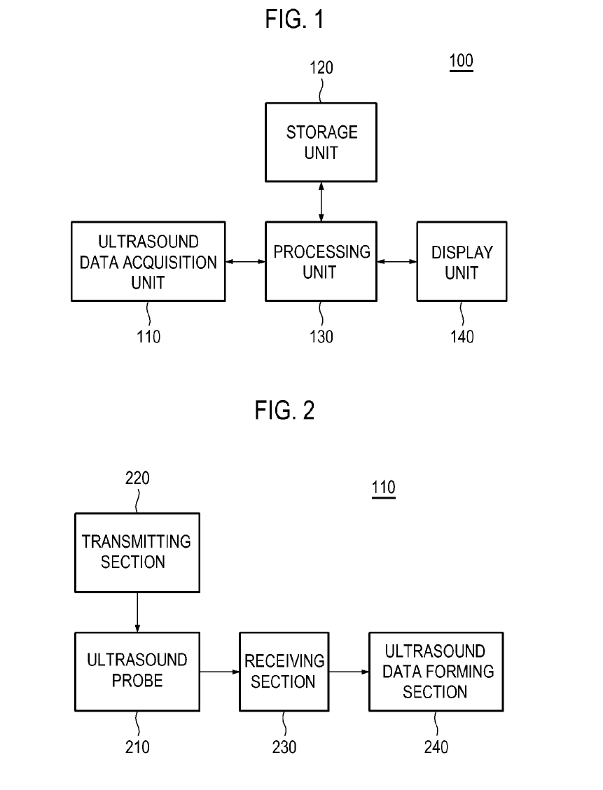

Referring to FIG. 1, an ultrasound system 100 in accordance with an illustrative embodiment is shown. As depicted therein, the ultrasound system 100 may include an ultrasound data acquisition unit 110.

The ultrasound data acquisition unit 110 may be configured to transmit ultrasound signals to a living body. The living body may include target objects (e.g., a heart, a liver, blood flow, a blood vessel, etc.). The ultrasound data acquisition unit 110 may be further configured to receive ultrasound signals (i.e., ultrasound echo signals) from the living body to acquire ultrasound data.

FIG. 2 is a block diagram showing an illustrative embodiment of the ultrasound data acquisition unit. Referring to FIG. 2, the ultrasound data acquisition unit 110 may include an ultrasound probe 210.

The ultrasound probe 210 may include a plurality of elements (not shown) for reciprocally converting between ultrasound signals and electrical signals. The ultrasound probe 210 may be configured to transmit the ultrasound signals to the living body. The ultrasound probe 210 may be further configured to receive the ultrasound echo signals from the living body to output electrical signals (“received signals”). The received signals may be analog signals. The ultrasound probe 210 may include a three-dimensional mechanical probe or a two-dimensional array probe. However, it should be noted herein that the ultrasound probe 210 may not be limited thereto.

The ultrasound data acquisition unit 110 may further include a transmitting section 220. The transmitting section 220 may be configured to control the transmission of the ultrasound signals. The transmitting section 220 may be further configured to generate electrical signals (“transmitting signals”) for obtaining an ultrasound image in consideration of the elements and focusing points. Thus, the ultrasound probe 210 may convert the transmitting signals into the ultrasound signals, transmit the ultrasound signals to the living body and receive the ultrasound echo signals from the living body to output the received signals.

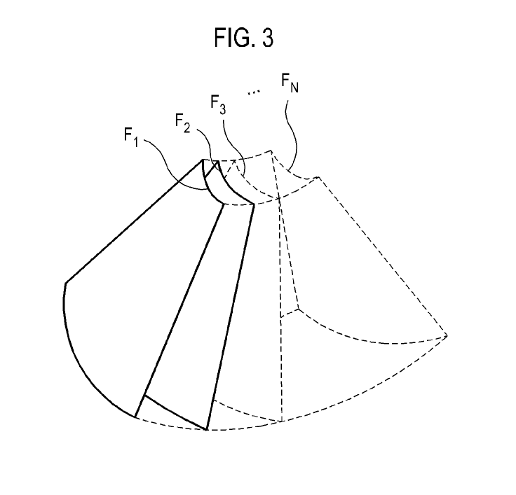

In one embodiment, the transmitting section 220 may generate the transmitting signals for obtaining a plurality of frames Fᵢ (1≦i≦N) corresponding to a three-dimensional ultrasound image at every predetermined time, as shown in FIG. 3. FIG. 3 is a schematic diagram showing an example of acquiring ultrasound data corresponding to the plurality of frames Fᵢ (1≦i≦N). The plurality of frames Fᵢ (1≦i≦N) may represent sectional planes of the living body (not shown).

Referring back to FIG. 2, the ultrasound data acquisition unit 110 may further include a receiving section 230. The receiving section 230 may be configured to convert the received signals provided from the ultrasound probe 210 into digital signals. The receiving section 230 may be further configured to apply delays to the digital signals in consideration of the elements and the focusing points to output digital receive-focused signals.

The ultrasound data acquisition unit 110 may further include an ultrasound data forming section 240. The ultrasound data forming section 240 may be configured to form ultrasound data based on the digital receive-focused signals provided from the receiving section 230. The ultrasound data may include radio frequency data. However, it should be noted herein that the ultrasound data may not be limited thereto.

In one embodiment, the ultrasound data forming section 240 may form the ultrasound data corresponding to each of frames Fᵢ (1≦i≦N) based on the digital receive-focused signals provided from the receiving section 230.

Referring back to FIG. 1, the ultrasound system 100 may further include a storage unit 120. The storage unit 120 may store the ultrasound data acquired by the ultrasound data acquisition unit 110. The storage unit 120 may further store a three-dimensional color reference table. The three-dimensional color reference table may be a table for providing colors corresponding to three-dimensional coordinates of a three-dimensional coordinate system that includes an X-axis of depth, a Y-axis of an intensity accumulation value and a Z-axis of a shading value.

The ultrasound system 100 may further include a processing unit 130 in communication with the ultrasound data acquisition unit 110 and the storage unit 120. The processing unit 130 may include a central processing unit, a microprocessor, a graphic processing unit and the like.

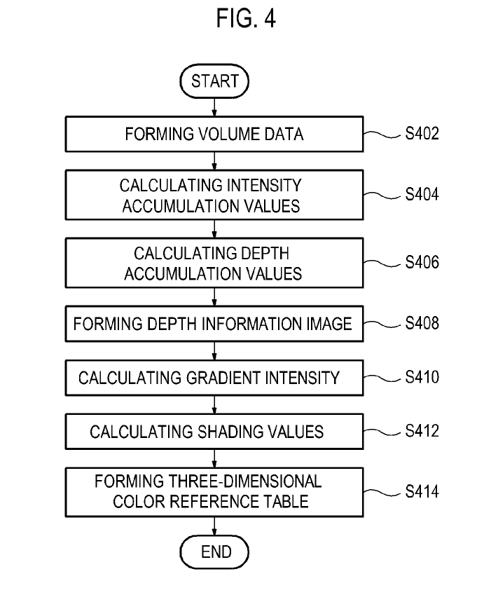

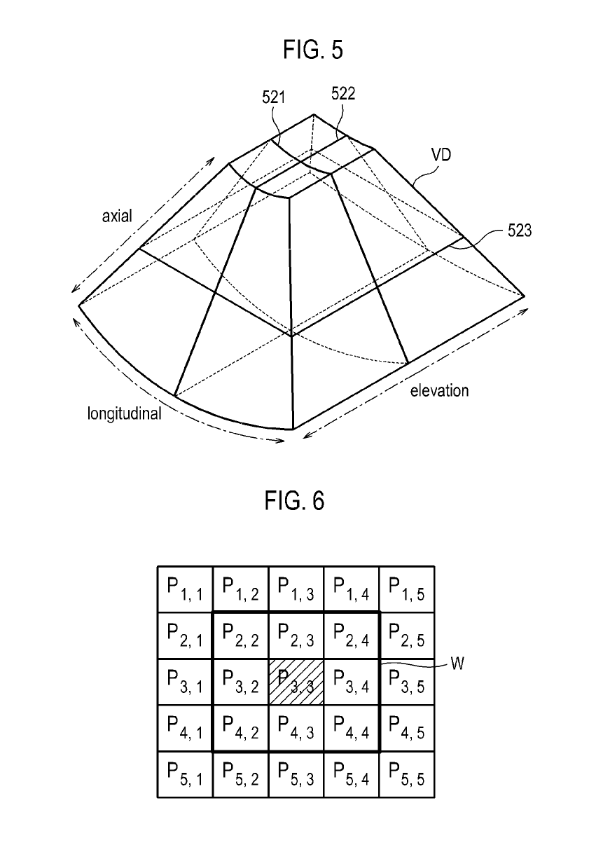

FIG. 4 is a flow chart showing a process of forming a three-dimensional color reference table. The processing unit 130 may be configured to synthesize the ultrasound data corresponding to each of the frames Fᵢ (1≦i≦N) to form volume data VD as shown in FIG. 5, at step S402 in FIG. 4.

FIG. 5 is a schematic diagram showing an example of the volume data. The volume data VD may include a plurality of voxels (not shown) having brightness values. In FIG. 5, reference numerals 521, 522 and 523 represent an A plane, a B plane and a C plane, respectively. The A plane 521, the B plane 522 and the C plane 523 may be mutually orthogonal. Also, in FIG. 5, the axial direction may be a transmitting direction of the ultrasound signals, the lateral direction may be a longitudinal direction of the elements, and the elevation direction may be a swing direction of the elements, i.e., a depth direction of the three-dimensional ultrasound image.

Referring back to FIG. 4, the processing unit 130 may be configured to perform volume-rendering upon the volume data VD to calculate intensity accumulation values throughout the depth, at step S404 in FIG. 4. Volume-rendering may include ray-casting for emitting virtual rays to the volume data VD.

In one embodiment, the processing unit 130 may accumulate intensity values of sample points on each of the virtual rays based on transparency (or opacity) of the sample points to calculate the intensity accumulation values throughout the depth as equation 1 provided below.

$${{\sum }_{i}}^{n} {I}_{i} {{\prod }_{J}}^{i - 1} {T}_{j} \qquad ( 1 )$$

In equation 1, I represents intensity, and T represents transparency.

The processing unit 130 may be configured to perform ray-casting upon the volume data VD to calculate depth accumulation values throughout the depth, at step S406 in FIG. 4. The processing unit 130 may be configured to form a depth information image based on the depth accumulation values, at step S408 in FIG. 4. The methods of forming the depth information image are well known in the art. Thus, they have not been described in detail so as not to unnecessarily obscure the present disclosure.

In one embodiment, the processing unit 130 may accumulate depth values of the sample points on each of the virtual rays based on transparency (or opacity) of the sample points to calculate the depth accumulation values throughout the depth as equation 2 provided below.

$${{\sum }_{i}}^{n} {D}_{i} {{\prod }_{j}}^{i - 1} {T}_{j} \qquad ( 2 )$$

In equation 2, D represents depth, and T represents transparency.

The processing unit 130 may be configured to calculate gradient intensity based on the depth information image, at step S410 in FIG. 4. Generally, the depth information image may be regarded as a surface having a height value corresponding to each of the pixels, and the gradient in the three-dimensional volume may be regarded as a normal of the surface.

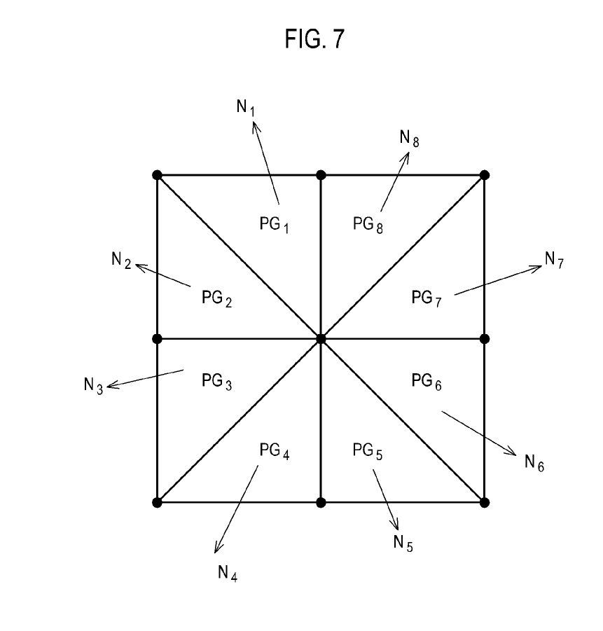

In one embodiment, the processing unit 130 may set a window W on the adjacent pixels P_{2,2}, P_{2,3}, P_{2,4}, P_{3,2}, P_{3,4}, P_{4,2}, P_{4,3} and P_{4,4} based on a pixel P_{3,3} as shown in FIG. 6. The processing unit 130 may further set a center point corresponding to each of pixels within the window Was shown in FIG. 7. The processing unit 130 may further set polygons PG₁ to PG₈ for connecting the adjacent pixels based on the center points. The processing unit 130 may further calculate normals N₁ to N₈ corresponding to the polygons PG₁ to PG₈. The methods of calculating the normal are well known in the art. Thus, they have not been described in detail so as not to unnecessarily obscure the present disclosure. The processing unit 130 may further calculate a mean normal of the calculated normals N₁ to N₈. The processing unit 130 may further set the calculated mean normal as the surface normal (i.e., gradient intensity) of the pixel P_{3,3}.

Although it is described that the processing unit 130 may set 8 pixels as the adjacent pixels based on the each of the pixels, the number of the adjacent pixels may not be limited thereto. Also, although it is described that the polygons for connecting the adjacent pixels are a triangle, the polygons may not be limited thereto.

The processing unit 130 may be configured to calculate shading values based on the surface normals and the virtual rays, at step S412 in FIG. 4. In one embodiment, the processing unit 130 may calculate scalar product values between vectors of the surface normals and vectors of the virtual rays as the shading values.

The processing unit 130 may be configured to form the three-dimensional color reference table based on the intensity accumulation values and the shading values, at step S414 in FIG. 4. In one embodiment, the processing unit 130 may form the three-dimensional color reference. The three-dimensional color reference table may be stored in the storage unit 120.

Optionally, the processing unit 130 may be configured to analyze the volume data VD to detect a skin tone of the target object (e.g., a fetus). The processing unit 130 may be further configured to apply the detected skin tone to the three-dimensional color reference table. The methods of detecting the skin tone are well known in the art. Thus, they have not been described in detail so as not to unnecessarily obscure the present disclosure.

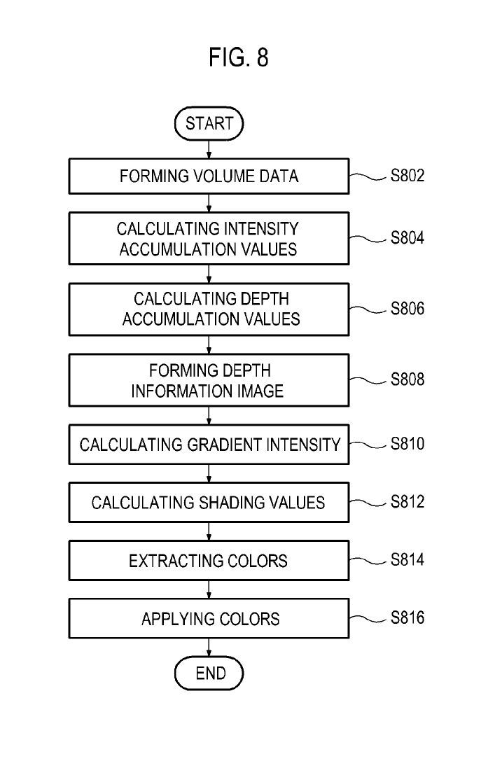

FIG. 8 is a flow chart showing a process of forming a three-dimensional ultrasound image. The processing unit 130 may be configured to form the volume data VD as shown in FIG. 5 based on the ultrasound data newly provided from the ultrasound data acquisition unit 110, at step S802 in FIG. 8.

The processing unit 130 may be configured to perform volume rendering (i.e., ray-casting) upon the volume data VD to calculate the intensity accumulation values throughout the depth, at step S804 in FIG. 8. The processing unit 130 may be configured to perform ray-casting upon the volume data VD to form the depth accumulation values throughout the depth, at step S806 in FIG. 8. The processing unit 130 may be configured to form the depth information image based on the depth accumulation values, at step S808 in FIG. 8. The processing unit 130 may be configured to the gradient intensity based on the depth information image, at step S810 in FIG. 8. The processing unit 130 may be configured to calculate the shading values based on the surface normals and the virtual rays, at step S812 in FIG. 8.

The processing unit 130 may be configured to retrieve the three-dimensional color reference table stored in the storage unit 120 to extract colors corresponding to the intensity accumulation values and the shading values throughout the depth, at step S814 in FIG. 8.

Optionally, the processing unit 130 may be configured to analyze the volume data VD to detect the skin tone of the target object (e.g., fetus). The processing unit 130 may be further configured to retrieve the three-dimensional color reference table to extract colors corresponding to the skin tone.

Also, the processing unit 130 may be configured to detect the skin tone (e.g., fetus) of the target object based on input information provided from a user input unit (not shown). The input information may be skin tone selection information for selecting the skin tone of parents or a race.

The processing unit 130 may be configured to apply the extracted colors to the volume data VD to form a three-dimensional ultrasound image, at step S816 in FIG. 8. In one embodiment, the processing unit 130 may apply the extracted colors to the voxels corresponding to the depth of the volume data VD to form the three-dimensional ultrasound image.

Referring back to FIG. 1, the ultrasound system 100 may further include a display unit 140. The display unit 140 may be configured to display the three-dimensional ultrasound image formed by the processing unit 130. The display unit 140 may include a cathode ray tube, a liquid crystal display, a light emitting diode, an organic light emitting diode and the like.

Although embodiments have been described with reference to a number of illustrative embodiments thereof, it should be understood that numerous other modifications and embodiments can be devised by those skilled in the art that will fall within the spirit and scope of the principles of this disclosure. More particularly, numerous variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. An ultrasound system, comprising:
a storage unit for storing a three-dimensional color reference table for providing colors corresponding to at least one of intensity accumulation values and shading values throughout depth; and
a processing unit configured to form volume data based on ultrasound data corresponding to a target object and perform ray-casting on the volume data to calculate intensity accumulation values and shading values throughout the depth, the processing unit being further configured to apply colors corresponding to the at least one of the calculated intensity accumulation values and the calculated shading values based on the three-dimensional color reference table.

2. The ultrasound system of claim 1, further comprising:
an ultrasound data acquisition unit configured to transmit ultrasound signals to a living body including the target object and receive ultrasound echo signals from the living body to acquire the ultrasound data.

3. The ultrasound system of claim 1, wherein the processing unit is configured to form the three-dimensional color reference table based on the volume data.

4. The ultrasound system of claim 3, wherein the processing unit is configured to:
perform ray-casting for emitting virtual rays upon volume data to calculate the intensity accumulation values and depth accumulation values throughout the depth;
form a depth information image based on the depth accumulation values;
calculate gradient intensity based on the depth information image;
calculate the shading values based on the gradient intensity; and
form the three-dimensional color reference table based on the depth, the intensity accumulation values and the shading values.

5. The ultrasound system of claim 4, wherein the processing unit is configured to:
set a window on adjacent pixels based on each of the pixels of the depth information image;
set a center point of each of the pixels within the windows;
set a plurality of polygons for connecting adjacent pixels based on the center points;
calculate a plurality of normals corresponding to the plurality of polygons;
calculate a mean normal of the normals; and
calculate the gradient intensity of a surface normal corresponding to each of the pixels of the depth information image based on the mean normal.

6. The ultrasound system of claim 5, wherein the processing unit is configured to calculate scalar product values between vectors of the surface normals and vectors of the virtual rays as the shading value.

7. The ultrasound system of claim 4, wherein the processing unit is further configured to:
analyze the volume data to detect a skin tone of the target object; and
apply the detected skin tone to the three-dimensional color reference table.

8. The ultrasound system of claim 1, wherein the processing unit is configured to:
perform ray-casting for emitting virtual rays upon the volume data to calculate the intensity accumulation values and depth accumulation values throughout the depth;
form a depth information image based on the depth accumulation values;
calculate gradient intensity based on the depth information image;
calculate the shading values based on the gradient intensity;
retrieve the three-dimensional color reference table to extract the colors corresponding to the intensity accumulation values and the shading values; and
apply the extracted colors to the volume data to form the three-dimensional ultrasound image.

9. The ultrasound system of claim 8, wherein the processing unit is configured to calculate scalar product values between vectors of the surface normals and vectors of the virtual rays as the shading value.

10. The ultrasound system of claim 8, wherein the processing unit is further configured to:
analyze the volume data to detect the skin tone of the target object;
retrieve the three-dimensional color reference table to extract colors corresponding to the skin tone; and
apply the extracted colors to the volume data.

11. A method of providing a three-dimensional ultrasound image, comprising:
a) forming volume data based on ultrasound data corresponding to a target object;
b) performing ray-casting on the volume data to calculate intensity accumulation values and shading values throughout the depth; and
c) applying colors corresponding to the at least one of the calculated intensity accumulation values and the calculated shading values based on a three-dimensional color reference table for providing colors corresponding to at least one of intensity accumulation values and shading values throughout depth.

12. The method of claim 11, wherein the step a) further comprises:
transmitting ultrasound signals to a living body including the target object; and
receiving ultrasound echo signals from the living body to acquire the ultrasound data.

13. The method of claim 11, further comprising:
forming the three-dimensional color reference table based on the volume data, prior to performing the step a).

14. The method of claim 13, wherein the step of forming the three-dimensional color reference table comprises:
performing ray-casting for emitting virtual rays upon volume data to calculate the intensity accumulation values and depth accumulation values throughout the depth;
forming a depth information image based on the depth accumulation values;
calculating gradient intensity based on the depth information image;
calculating the shading values based on the gradient intensity; and
forming the three-dimensional color reference table based on the depth, the intensity accumulation values and the shading values.

15. The method of claim 14, wherein calculating gradient intensities comprises:
setting a window on adjacent pixels based on each of pixels of the depth information image;
setting a center point of each of the pixels within the windows;
setting a plurality of polygons for connecting adjacent pixels based on the center points;
calculating a plurality of normals corresponding to the plurality of polygons;
calculating a mean normal of the normals; and
calculating the gradient intensity of a surface normal corresponding to each of the pixels of the depth information image based on the mean normal.

16. The method of claim 14, wherein calculating the shading values comprises:
calculating scalar product values between vectors of the surface normals and vectors of the virtual rays as the shading value.

17. The method of claim 14, wherein the step of forming the three-dimensional color reference table further comprises:
analyzing the volume data to detect a skin tone of the target object; and
applying the detected skin tone to the three-dimensional color reference table.

18. The method of claim 11, wherein the step c) comprises:
c1) performing ray-casting for emitting virtual rays upon the volume data to calculate the intensity accumulation values and depth accumulation values throughout the depth;
c2) forming a depth information image based on the depth accumulation values;
c3) calculating gradient intensity based on the depth information image;
c4) calculating the shading values based on the gradient intensity;
c5) retrieving the three-dimensional color reference table to extract the colors corresponding to the intensity accumulation values and the shading values; and
c6) applying the extracted colors to the volume data to form the three-dimensional ultrasound image.

19. The method of claim 18, wherein the step c4) comprises:
calculating scalar product values between vectors of the surface normals and vectors of the virtual rays as the shading value.

20. The method of claim 19, wherein the step c) further comprises:
analyzing the volume data to detect the skin tone of the target object;
retrieving the three-dimensional color reference table to extract colors corresponding to the skin tone; and
applying the extracted colors to the volume data.
